(19) United States
(12) Patent Application Publication
(10) Pub. No.: US 2025/0335747 A1
(43) Pub. Date: Oct. 30, 2025

(54) Method and Device for Analyzing Multi-Channel Time Series Signals Using a Deep Learning Model

(71) Applicant: Robert Bosch GmbH, Stuttgart (DE)

(72) Inventors: Zhigang Sun, Shanghai (CN); Yifei Yu, Shanghai (CN); Shouyi Zhang, Shanghai (CN); Lijuan Xia, Shanghai (CN)

(21) Appl. No.: 19/190,698

(22) Filed: Apr. 27, 2025

(30) Foreign Application Priority Data
Apr. 29, 2024 (CN) 2024 1052 8685.0

(51) Int. Cl. G06N 3/0464; B60W 40/08; B60W 50/00; G06N 3/045

(52) U.S. Cl. CPC G06N 3/0464; B60W 40/08; B60W 50/0097; G06N 3/045; B60W 2040/0818; B60W 2540/229

(57) ABSTRACT

A method for analyzing multi-channel time series signals using a deep learning model includes (i) obtaining the multi-channel time series signals, and (ii) using the deep learning model to generate a model prediction value based on the multi-channel time series signals. The deep learning model includes a convolutional neural network module and a transformer module. The convolutional neural network module is configured to receive the multi-channel time series signals and generate a convolutional output. The transformer module is configured to receive the convolutional output and generate the model prediction value. A method for controlling a vehicle includes (i) obtaining a model prediction value generated according to the above analysis method, and (ii) generating instructions based on the model prediction value for triggering an autonomous driving control unit of the vehicle to perform an autonomous driving operation.

### BACKGROUND

This application claims priority under 35 U.S.C. § 119 to application no. CN 2024 1052 8685.0, filed on Apr. 29, 2024 in China, the disclosure of which is incorporated herein by reference in its entirety.

The present disclosure relates generally to the computer field and more particularly to a method and a device for analyzing multi-channel time series signals using a deep learning model.

The purpose of signal analysis is to extract the effective information carried by the signals. In recent years, as machine learning technology continues to evolve, various machine learning models have been able to provide increasingly robust data processing capabilities. In this context, it has been proposed that machine learning models can be used to replace traditional mathematical operations to perform signal analysis.

Multi-channel time series signals are signals carrying complex information. In signal analysis of multi-channel time series signals using conventional machine learning models, manual feature extraction is typically required for the original multi-channel time series signals first and then the extracted feature data are provided to the machine learning model so as to further capture the effective information in the signals. However, manual feature extraction has adverse effects in some aspects, such as low efficiency, being limited by the knowledge of the person performing the feature extraction, and so on. These effects may further result in unsatisfactory accuracy of the predicted results generated by the machine learning model. Therefore, there is a need for an improved method to more accurately and efficiently analyze multi-channel time series signals.

### SUMMARY

The present disclosure provides an improved mechanism for analyzing multi-channel time series signals using a deep learning model, which can be used in an end-to-end manner to automatically generate a model prediction value indicating effective information in multi-channel time series signals based on the original multi-channel time series signals without performing the manual feature extraction operations required in conventional machine learning methods.

According to one aspect of the present disclosure, a method is provided for analyzing multi-channel time series signals using a deep learning model, comprising: obtaining the multi-channel time series signals; and using the deep learning model to generate a model prediction value based on the multi-channel time series signals; wherein: the deep learning model comprises a convolutional neural network module and a transformer module; the convolutional neural network module is configured to receive the multi-channel time series signals and generate a convolutional output; and the transformer module is configured to receive the convolutional output and generate the model prediction value.

According to another aspect of the present disclosure, a method is provided for control of a vehicle, comprising: obtaining a model prediction value generated according to the above analysis method; and generating instructions based on the model prediction value for triggering an autonomous driving control unit of the vehicle to perform an autonomous driving operation.

According to another aspect of the present disclosure, a device is provided for processing multi-channel time series signals comprising: a memory and a processor. The processor is coupled with the memory and is configured to perform the method according to any one of various examples of the present disclosure.

According to still another aspect of the present disclosure, a computer-readable medium is provided storing a computer program comprising instructions, the instructions, when executed by a processor, causing the processor to be configured to perform the method according to any one of various examples of the present disclosure.

According to yet another aspect of the present disclosure, a computer program product is provided that includes computer executable instructions that, when executed, cause one or more processors to perform the method according to any one of various examples of the present disclosure.

### BRIEF DESCRIPTION OF THE DRAWINGS

The various examples of the subject matter to be protected are described by way of typical examples with reference to the accompanying drawings. The same reference signs are used in different accompanying drawings to denote the same or similar components.

FIG. 1 shows a schematic diagram of the principles of using conventional machine learning techniques to analyze multi-channel time series signals.

FIG. 2 shows a schematic diagram of the principles of using a deep learning model to analyze multi-channel time series signals in an end-to-end manner according to one example of the present disclosure.

FIG. 3 shows a schematic diagram of the structure of the convolutional neural network module of a deep learning model according to one example of the present disclosure.

FIG. 4 shows a schematic diagram of the structure of the transformer module of a deep learning model according to one example of the present disclosure.

FIG. 5 shows a schematic diagram of the structure of a transformer module comprising a plurality of stacked encoders and decoders according to one example of the present disclosure.

FIG. 6 shows an example flowchart of the method of using a deep learning model to analyze multi-channel time series signals according to one example of the present disclosure.

FIG. 7 shows an example flowchart of a method for controlling a vehicle according to one example of the present disclosure.

FIG. 8 shows a block diagram of a device for processing multi-channel time series signals according to one example of the present disclosure.

### DETAILED DESCRIPTION

In the following description, numerous specific details are set forth to provide a thorough understanding of the examples of the present disclosure. However, those skilled in the relevant art will recognize that the present disclosure can be practiced without one or more of the specific details, or by using alternative methods, components, etc., to practice the present disclosure. In some instances, well-known structures and operations are not shown or described in detail to avoid unnecessarily obscuring the present disclosure.

A time series signal refers to a data series formed over time by signal data over a period of time. In some scenarios, a time series signal may be acquired simultaneously from a plurality of different spatial locations to better characterize the spatial characteristics of the signal. These spatial locations generally correspond to different data channels, and therefore, such time series signals, which are acquired in a multi-channel manner, characterizing the spatial features, may be referred to as multi-channel time series signals.

One type of typical multi-channel time series signals comprises EEG signals. EEG signals, also known as electroencephalography (EEG), are electrical signals used to characterize the activity of brain neurons. Brain neurons form complex neural networks by synapse linkage to each other. Bioelectric phenomena occur when neurons are activated. Therefore, the electrical signals generated by the activation of neurons, that is, EEG signals, can be captured by electrodes placed on the scalp or directly implanted in the brain. EEG signals can generally be acquired from different positions on the subject's head so that the EEG signals can convey information about the brain's activity status more comprehensively and accurately.

In addition to the EEG signals discussed above, other common multi-channel time series signals may include various biological signals, such as ECG signals, various industrial signals such as mechanical vibration signals, and so on.

It will be understood from the above discussion that the multi-channel time series signals contain both temporal features characteristic of the time series signal and spatial features introduced by the multi-channel acquisition mode. Thus, it is generally challenging to analyze multi-channel time series signals to accurately and efficiently capture the effective information contained therein.

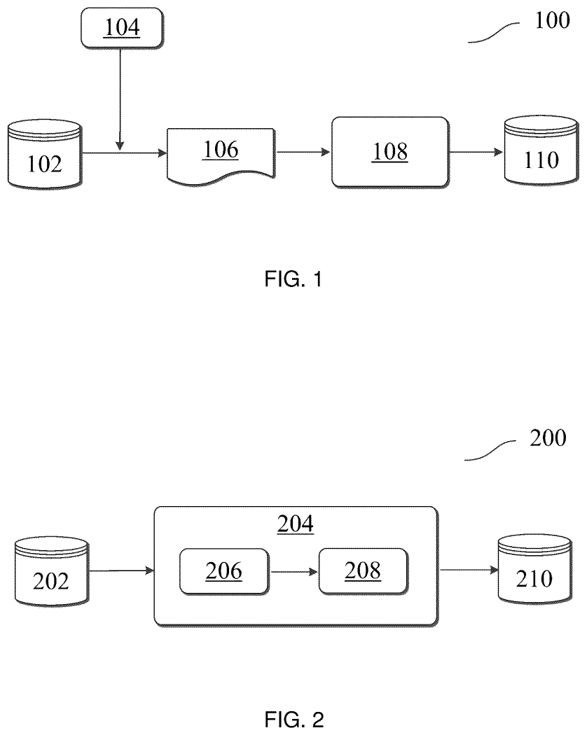

Currently, it has been proposed that the signal analysis of multi-channel time series signals can be performed using machine learning models instead of traditional mathematical operations. FIG. 1 shows a schematic diagram 100 of the principles of using conventional machine learning techniques to analyze multi-channel time series signals.

As shown in FIG. 1, multi-channel time series signals 102 can be obtained.

Manual feature extraction 104 may be performed on the multi-channel time series signals 102 in order to generate feature data 106. Feature extraction refers to the process of converting raw data into features that are representative and interpretable, which can help identify effective information from the raw data. Feature extraction may result in a decrease in the data dimension while retaining the effective information in the raw data, thereby contributing to improved computational efficiency and improved performance of the machine learning model. Manual feature extraction refers to the process of manually extracting feature data from raw data. Typically, feature data that can be manually extracted from raw data include statistical features, frequency domain features, time domain features, and so on. For example, in performing signal analysis, common manually extracted features may include power spectral density (PSD), differential entropy (DE), and so on. Of these, power spectral density is a physical quantity that describes how the power of the time series is distributed with frequency and differential entropy is a physical quality that describes the degree of randomness of a signal distribution.

The extracted feature data 106 may be provided to the machine learning model 108 in order to generate a model prediction value 110. The machine learning model 108 may be a conventional machine learning model such as a support vector machine (SVM), decision tree (DT), random forest (RF), etc., a deep learning model such as a convolutional neural network (CNN), a long short-term memory network (LSTM), a recurrent neural network (RNN), etc., or other models. The model prediction value 110 produced by the machine learning model 108 may indicate a result of signal analysis performed on the multi-channel time series signals 102.

In a scheme using traditional machine learning techniques to analyze multi-channel time series signals discussed in conjunction with FIG. 1, performing the operations of manual feature extraction 104 is essential. This is because the network structure of traditional machine learning models is generally relatively simple, while the ability of simple network structures to capture complex and dynamic input-output relationships is often limited. Thus, there is a need to initially filter out the more important features through manual feature extraction operations.

However, manual feature extraction may have adverse effects in some aspects. On the one hand, manual feature extraction generally relies heavily on the expertise of expert personnel performing feature extraction operations. Different expert personnel may extract different features or use different feature extraction methods, which may lead to differences in model prediction results. On the other hand, manual feature extraction relies on prior knowledge of the relevant features of a model task; in other words, the features that are manually extracted are limited to known features, such as the power spectral density and differential entropy discussed above, among others. This may result in missing important features that are not known a priori, thus affecting the accuracy of the model's prediction structure. In additional, manual feature extraction often requires different features for different tasks, so when a task changes, the feature extraction method may need to be modified, reducing flexibility.

In response to the above issues, this disclosure provides an improved mechanism for analyzing multi-channel time series signals using a deep learning model. The deep learning model of the present disclosure includes a convolutional neural network module and a transformer module in order to better automatically capture the spatial and temporal characteristics of the multi-channel time series signals. As such, the mechanism proposed by the present disclosure can use an end-to-end manner to automatically generate a model prediction value indicating effective information in multi-channel time series signals based on the original multi-channel time series signals without performing the manual feature extraction operations required in conventional machine learning methods.

FIG. 2 shows a schematic diagram 200 of the principles of using a deep learning model to analyze multi-channel time series signals in an end-to-end manner according to one example of the present disclosure.

For clarity, the mechanism proposed by the present disclosure for analyzing multi-channel time series signals using a deep learning model is discussed below in conjunction with an example application scenario. In this example application scenario, the deep learning model of the present disclosure may be utilized to perform the task of estimating the level of alertness of a driver based on EEG signals of a driver of a vehicle. Driver alertness estimation is an important field of research in autonomous driving technology that aims to identify whether the driver is in a state such as falling asleep or losing focus on the driving environment while driving so that corresponding strategies can be formulated in a timely manner, such as activating various autonomous driving technologies to avoid dangerous situations. Driver alertness estimation tasks are therefore critical to improving road safety.

Referring to FIG. 2, multi-channel time series signals 202 may be obtained. In one example, such as the example application scenario of performing the driver alertness estimation task discussed above, the multi-channel time series signals 202 may be EEG signals acquired from different positions of the head of a driver of a vehicle. For example, in one example scenario, the driver of the vehicle may be made to wear an EEG signal acquisition device, such as a helmet or other device containing an EEG data sensor. The EEG signal acquisition device can collect EEG data from different positions of the driver's head, such as the forehead, the back of the head, the left side of the forehead, the right side of the forehead, or other positions, thereby generating multi-channel time series signals 202. The data of each channel in the multi-channel time series signals 202 is a series of EEG data changing at multiple sampling time instants.

The original multi-channel time series signals 202 may be provided directly to the deep learning model 204. The deep learning model 204 may comprise a convolutional neural network module 206 and a transformer module 208, and the convolutional neural network module 206 and the transformer module 208 may be connected in series. The convolutional neural network module 206 may be configured to receive the multi-channel time series signals 202 and generate a convolutional output. The transformer module 208 may be configured to receive a convolutional output generated by the convolutional neural network module 206 and generate a model prediction value 210.

It is advantageous to combine the convolutional neural network module 206 and the transformer module 208 to form a deep learning model 204 to perform the analysis of the multi-channel time series signals 202.

As mentioned above, the multi-channel time series signals 202 is a particular complex signal with both spatial and temporal features. The spatial features are associated with the spatial relationship between the various data of the multi-channel time series signals 202, and such a spatial relationship is typically introduced by a multi-channel acquisition method. For example, the spatial relationship may be a relationship between data corresponding to an EEG signal acquisition position on the left side of the forehead and data corresponding to an EEG signal acquisition position on the right side of the forehead. The time features are associated with the temporal relationship between the various data of the multi-channel time series signals 202, and such a temporal relationship is typically inherent to the time series signal. For example, the temporal relationship may be a sequential relationship between a plurality of sampling time instants for which data are obtained from one data channel of an EEG signal.

The convolutional neural network module 206 is adapted to automatically extract a variety of complex spatial and temporal features of the multi-channel time series signals 202 associated with the task being performed. The transformer module 208 uses an attention mechanism. The transformer module 208 may combine the various feature data extracted by the convolutional neural network module 206 and automatically learn which features are more important and which features are less important in order to focus attention on the more important feature data. As such, the deep learning model 204 formed by the combination of the convolutional neural network module 206 and the transformer module 208 can accurately capture the effective information carried by the multi-channel time series signals 202.

Moreover, since the transformer module 208 itself has many parameters, it is usually computationally expensive and the training process is also complicated. According to the mechanism of the present disclosure, a convolutional neural network module 206 is connected in series before the transformer module 208. In this instance, the convolutional neural network module 206 can first extract a portion of the features from the original multi-channel time series signals 202 and thus have the effect of data dimensionality reduction. This helps reduce the number of parameters of the transformer module 208. As such, the deep learning model 204 formed by the combination of the convolutional neural network module 206 and the transformer module 208 has improved model computational efficiency and reduced model training cost and difficulty.

The model prediction value 210 generated by the deep learning model 204 based on the original multi-channel time series signals 202 may indicate effective information contained in the multi-channel time series signals 202. In one example, such as the example application scenario of performing the driver alertness estimation task discussed above, the model prediction value 210 may be a predicted regression value indicative of the driver's level of alertness. For example, the model prediction value 210 may be a value with a value ranging from 0-1, with lower values represents lower levels of driver alertness. In one example, the model prediction value 210 may also be a predicted classification value indicative of the level of alertness of the driver. For example, the model prediction value 210 may be 0 to indicate that the driver is currently not alert. Similarly, the model prediction value 210 may be 1 to indicate that the driver is currently alert, and so on. It will be understood that the model prediction value 210 generated by the deep learning model 204 may have different representations depending on which training data are used to train the deep learning model 204.

While the principles of the mechanism of the present disclosure are discussed in the above discussion with the driver alertness estimation task as an example, it should be understood that any other tasks may be performed using the mechanism discussed in the present disclosure. Depending on the specific task performed, the input of the deep learning model may be other multi-channel time series signals that differ from the driver EEG signals discussed above, and the output of the deep learning model may be other model prediction values that differ from the driver alertness level discussed above.

As such, according to the mechanism of the present disclosure, the original multi-channel time series signals may be input directly into the deep learning model to generate a model prediction value without manual feature extraction operations. That is, the deep learning model of the present disclosure automatically learns and extracts features related to the task performed from the original multi-channel time series signals in an end-to-end pipeline and generates accurate prediction results.

This end-to-end approach to deep learning has a range of advantages over traditional approaches that include manual feature extraction operations. On the one hand, the end-to-end deep learning method of the present disclosure avoids relying on the knowledge of the person performing the manual feature extraction. This makes the model prediction process less susceptible to human bias, which helps to improve the consistency and reproducibility of model prediction results. Further, the above approach helps the deep learning model to automatically learn various types of data features from the original multi-channel time series signals, particularly those that are currently not well known, thereby further improving the accuracy of model prediction results. On the other hand, the end-to-end deep learning method of the present disclosure avoids the issue of manual feature extraction requiring adjustment for different raw data and tasks, but can better adapt to changes in raw data or tasks and is therefore highly flexible.

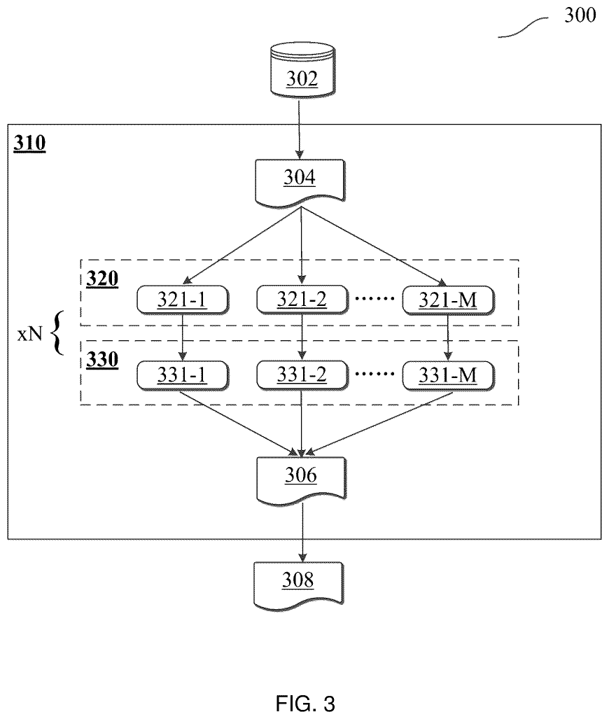

FIG. 3 shows a schematic diagram 300 of the structure of the convolutional neural network module of a deep learning model according to one example of the present disclosure. As shown in FIG. 3, the convolutional neural network module 310 may receive the original multi-channel time series signals 302 and generate a convolutional output 308. In one example, the convolutional neural network module 310 shown in FIG. 3 may correspond to the convolutional neural network module 206 discussed above in conjunction with FIG. 2.

The convolutional neural network module 310 may be configured to first shape the multi-channel time series signals 302 to generate shaped two-dimensional input data 304. The multi-channel time series signals 302 comprises data from a plurality of channels, and the data from each channel is one-dimensional, i.e., a one-dimensional time series signal that varies over a plurality of sampling time instants. The shaping performed by the convolutional neural network module 310 may shape a one-dimensional time series signal for a plurality of channels into two-dimensional data, for example, data similar to an image format. Any known signal shaping algorithms can be used to perform the above shaping operation, e.g., Gramian angular field (GAF), Markov transition field (MTF), short-time Fourier transform (STFT), etc. The shaping of the multi-channel time series signals 302 into two-dimensional input data 304 facilitates subsequent performance of two-dimensional convolutional operations. By way of two-dimensional convolutional operations, the spatial features of the multi-channel time series signals 302 can be better captured, thereby helping to improve the overall performance of the deep learning model.

The shaped two-dimensional input data 304 may be input into the network portion of the convolutional neural network module 310. As shown in FIG. 3, the network portion of the convolutional neural network module 310 may comprise a convolutional layer 320 for implementing convolutional operations and a pooling layer 330 for implementing pooling operations. While only one convolutional layer 320 and one pooling layer 330 are clearly illustrated in FIG. 3, in one example, the network portion of the convolutional neural network module 310 may be formed by N alternating convolutional layers and N corresponding pooling layers. That is, the structure of the network portion of the convolutional neural network module 310 may be a first convolutional layer, a first pooling layer, a second convolutional layer, a second pooling layer . . . an Nth convolutional layer, and an Nth pooling layer.

As shown in FIG. 3, the shaped two-dimensional input data 304 is used as an input to the first convolutional layer (e.g., convolutional layer 320) of the N convolutional layers. The convolutional layer 320 may comprise a plurality of convolutional units 321. The convolutional unit 321 may also be referred to as a convolutional kernel or filter that may perform a convolutional operation on the shaped two-dimensional input data 304 in order to capture the various spatial and temporal features contained in the multi-channel time series signals 302. Different parameters may be set for each convolutional unit 321, such as size of convolutional kernel, weight value, step size, etc., such that each convolutional unit 321 captures a class of spatial or temporal features.

The pooling layer 330 may receive an output from the convolutional layer located on layer above it in order to implement a pooling operation. The pooling layer 330 may comprise a plurality of pooling units 331. In one example, the number of pooling units 331 may be consistent with the number of convolutional units contained in the convolutional layer located above it. The pooling unit 331 may achieve downsampling of the feature data output from the convolutional layer using, for example, a maximum or average pooling algorithm to achieve a significant reduction in the amount of data while retaining effective features as much as possible.

The convolutional neural network module 310 may also be configured to shape the output 306 of the last of the N pooling layers. The shaping operation may be the inverse operation of the above operation of shaping the one-dimensional time series signal for a plurality of channels into two-dimensional data, thereby causing the convolutional neural network module 310 to generate a one-dimensional convolution output 308. By using the shaping operation to make the convolution output 308 into one-dimensional data, it is more conducive to the subsequent data processing by the transformer module connected after the convolutional neural network module 310. For example, this allows the transformer module to assign a corresponding attention to each data point in the one-dimensional convolutional output in order to automatically learn which features are more important.

The one-dimensional convolutional output 308 generated by the convolutional neural network module 310 is a series of convolutional values corresponding to a plurality of time instants. These time instants are also the plurality of sampling time instants of the multi-channel time series signals mentioned above. One or more convolutional values in the series may correspond to each of the plurality of time instants.

It will be understood that the structure of the convolutional neural network module discussed in conjunction with FIG. 3 is merely one example, and in other examples, other structures may be employed to achieve the above operations.

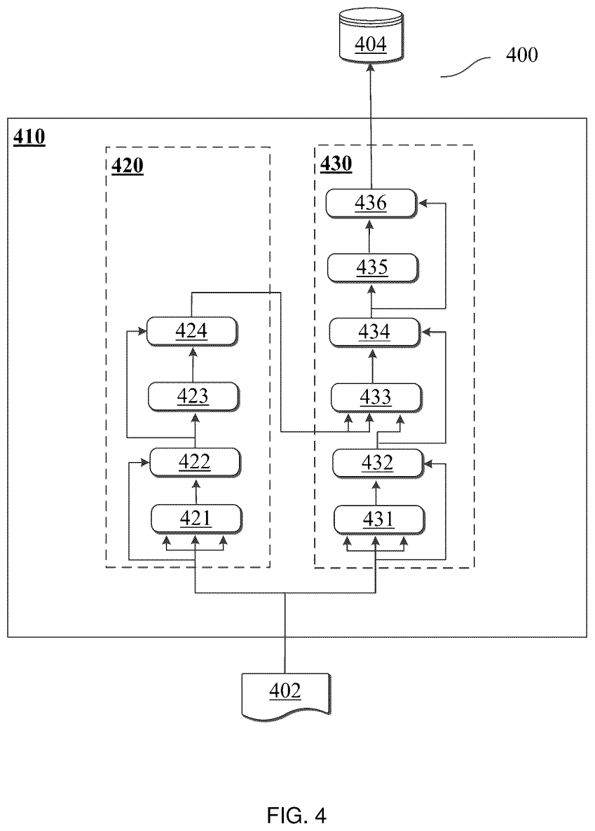

FIG. 4 shows a schematic diagram 400 of the structure of the transformer module of a deep learning model according to one example of the present disclosure. As shown in FIG. 4, the transformer module 410 may receive the convolutional output 402 (which may correspond to the convolutional output 308 discussed above in conjunction with FIG. 3) and generate a model prediction value 404. In one example, the transformer module 410 shown in FIG. 4 may correspond to the transformer module 208 discussed above in conjunction with FIG. 2.

The transformer module 410 has an encoder-decoder architecture. In one example, the transformer module 410 may comprise an encoder 420 and a corresponding decoder 430.

The transformer module 410 employs an attention mechanism to automatically learn which portions of the feature data are more important or more recognizable in order to assign higher attention to the more important features. The attention mechanism may be implemented primarily through the attention units contained in the encoder 420 and decoder 430 discussed below.

The encoder 420 may comprise an encoder attention unit. The encoder attention unit is configured to receive a convolutional value in the convolutional output 402 corresponding to a first time instant of the plurality of time instants. As shown in FIG. 4, the encoder attention unit may comprise a multi-head attention sublayer 421 and a residual connection and normalization sublayer 422. The multi-head attention sublayer 421 is used to automatically capture different features for a plurality of linear subspaces of the input data (e.g., the convolutional value corresponding to the first time instant). The residual connection and normalization sublayer 422 is used to perform skip residual operations and normalization operations on the input and output of the multi-head attention sublayer 421.

The encoder 420 also comprises an encoder feedforward unit, and the encoder feedforward unit is connected behind the encoder attention unit. The encoder feedforward unit is configured to receive an output of the encoder attention unit and generate an encoder output for the first time instant. As shown in FIG. 4, the encoder feedforward unit may comprise a feedforward sublayer 423 and a residual connection and normalization sublayer 424. The feedforward sublayer 423 may be implemented as a fully connected network with two linear layers to improve the fit of the attention mechanism for complex processes. The residual connection and normalization sublayer 424 may perform similar operations to those discussed above for the residual connection and normalization sublayer 422, such as skip residual operations and normalization operations on the input and output of the feedforward sublayer 423.

The decoder 430 may the receive the convolutional output 402 and the output of the encoder 420 simultaneously to generate a decoder output. The decoder 430 may comprise a masked attention unit. The masked attention unit is configured to receive a convolutional value in the convolutional output 402 corresponding to a second time instant of a plurality of time instants, wherein the first time instant is before the second time instant. In one example, the second time instant may be a time instant to be predicted, in other words, the current time instant to perform the prediction task, while the first time instant may be the time instant before the current time instant. In other words, the transformer module 410 may utilize historical data from previous time instants to generate a prediction for the time instant. As shown in FIG. 4, the masked attention unit may comprise a masked multi-head attention sublayer 431 and a residual connection and normalization sublayer 432. The masked multi-head attention sublayer 431 works in a similar way to the multi-head attention sublayer 421 discussed above, but further enables masking functions to avoid early data leakage after the current time instant during the model training process stage, thereby expediting the training process. The residual connection and normalization sublayer 432 may perform similar operations to those discussed above for the residual connection and normalization sublayer 422, such as skip residual operations and normalization operations on the input and output of the masked multi-head attention sublayer 431.

The decoder 430 also comprises a decoder attention unit that is connected after the masked attention unit.

The decoder attention unit is configured to receive the output of the masked attention unit and the encoder output generated by the encoder 420 for the first time instant. As shown in FIG. 4, the decoder attention unit may comprise a multi-head attention sublayer 433 and a residual connection and normalization sublayer 434. The multi-head attention sublayer 433 and the residual connection and normalization sublayer 434 may work in a similar way to the multi-head attention sublayer 421 and the residual junction and normalization sublayer 422 discussed above, respectively.

The decoder 430 also comprises a decoder feedforward unit, and the decoder feedforward unit is connected behind the decoder attention unit. The decoder feedforward unit is configured to receive an output of the decoder attention unit and generate a decoder output for the second time instant. As shown in FIG. 4, the decoder feedforward unit may comprise a feedforward sublayer 435 and a residual connection and normalization sublayer 436. The feedforward sublayer 435 and the residual connection and normalization sublayer 436 may work in a similar way to the feedforward sublayer 423 and the residual junction and normalization sublayer 424 discussed above, respectively.

The decoder 430 may generate a decoder output, and the decoder output is for the second time instant. In one example, the output of the residual connection and normalization sublayer 436 shown in FIG. 4 may be directly taken as the output 404 of the deep learning model when performing a regression task with the deep learning model to generate a regression value, e.g., one indicating the level of alertness of a driver.

In one example, the output of the residual connection and normalization sublayer 436 may be further processed to generate a model prediction value. In this example, the decoder 430 may also comprise other structures not shown in FIG. 4. For example, if the task performed with the deep learning model is a classification task rather than a regression task, additional linear sublayers and classification normalization sublayers may be attached after the residual connection and normalization sublayer 436. The attached linear sublayer is used to perform a linear transformation operation on the output of the previous layer to obtain the output of the specified dimension. The attached classification normalization sublayer is used to cause the generated model prediction value to fall within the probability threshold of [0,1] in order to [obtain] a classification result, which can be achieved by a classification normalization function such as softmax.

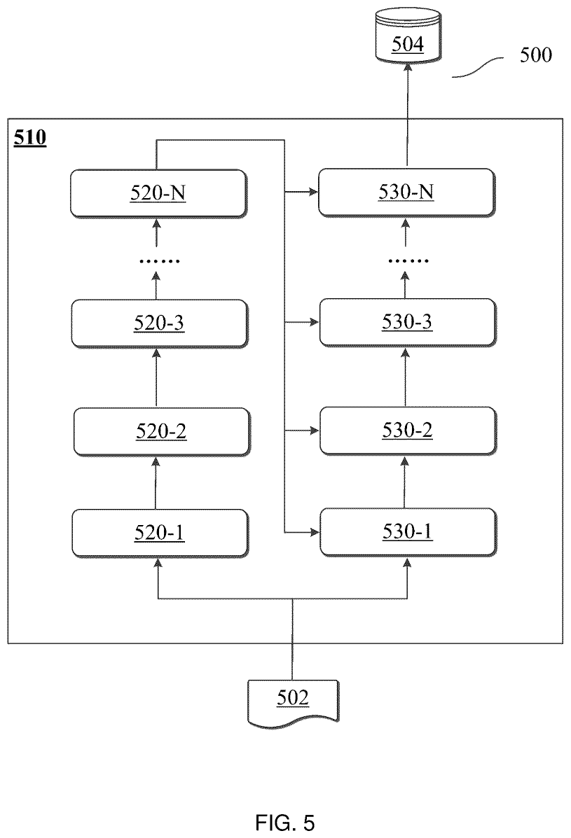

Although FIG. 4 only exemplarily shows that the transformer module 410 comprises one encoder 420 and one decoder 430, in another example, the transformer module 410 may comprise a plurality of stacked encoders and a plurality of corresponding stacked decoders. FIG. 5 shows a schematic diagram 500 of the structure of a transformer module comprising a plurality of stacked encoders and decoders according to one example of the present disclosure.

FIG. 5 shows a transformer module 510 that comprises N stacked encoders 520-1 to 520-N and N stacked decoders 530-1 to 530-N. The transformer module 510 may receive the convolutional output 502 and generate a model prediction value 504. The convolutional output 502 may be similar to the convolutional output 402 discussed above in conjunction with FIG. 4 and the model prediction value 504 may be similar to the model prediction value 404 discussed above in conjunction with FIG. 4. Thus, the convolutional output 502 and the model prediction value 504 will not be discussed in detail here.

The N stacked encoders 520-1 to 520-N can be connected in series. In the present disclosure, for clarity, encoders 520-1 to 520-N are referred to separately as the first encoder 520-1, the second encoder 520-2, and so on, until the last encoder 520-N. The first encoder 520-1 may use the convolutional output 502 as input and may generate a first encoder output. Each encoder from the second encoder 520-2 to the last encoder 520-N can use the encoder output generated by the previous encoder as input and generate a corresponding encoder output. For example, the second encoder 520-2 can use the first encoder output generated by the first encoder 520-1 as input and generate a second encoder output, and so on.

Each encoder from encoders 520-1 to 520-N may have a similar structure to that of encoder 420 discussed above in conjunction with FIG. 4. Each encoder may comprise an encoder attention unit and an encoder feedforward unit. For example, the first encoder 520-1 may comprise a first encoder attention unit and a first encoder feedforward unit, and the second encoder 520-2 may comprise a second encoder attention unit and a second encoder feedforward unit, and so on.

For the first encoder 520-1, the first encoder attention unit may receive the convolutional value corresponding to the first time instant in the convolutional output 502 and the first encoder feedforward unit may receive the output of the first encoder attention unit and generate a corresponding encoder output for the first time instant.

For each of the second to last encoders 520-2 to 520-N, the encoder attention unit of the encoder can receive the encoder output generated by the previous encoder and the encoder feedforward unit of the encoder can receive the output of the encoder attention unit and generate a corresponding encoder output for the first time instant.

The N stacked decoders 530-1 to 530-N can also be connected in series. In the present disclosure, for clarity, decoders 530-1 to 530-N are referred to separately as the first decoder 530-1, the second decoder 530-2, and so on, until the last decoder 530-N. The first decoder 520-1 may use the convolutional output 502 and the encoder output generated by the last encoder 520-N as input and may generate a first decoder output. Each decoder from the second to the last decoders 530-2 to 530-N may use the encoder output generated by the last encoder 520-N and the decoder output generated by the previous decoder as input and generate a corresponding decoder output. For example, the second decoder 530-2 may use the encoder output generated by the last encoder 520-N and the decoder output generated by the first decoder 530-1 as input and generate a corresponding decoder output, and so on.

Each decoder from decoders 530-1 to 530-N may have a similar structure to that of decoder 430 discussed above in conjunction with FIG. 4. Each decoder may comprise a masked attention unit, a decoder attention unit, and a decoder feedforward unit. For example, the first decoder 530-1 may comprise a first masked attention unit, a first decoder attention unit, and a first decoder feedforward unit, and the second decoder 530-2 may comprise a second masked attention unit, a second decoder attention unit, and a second decoder feedforward unit, and so on.

For the first decoder 530-1, the first masked attention unit may receive the convolutional value corresponding to the second time instant in the convolutional output 502, the first decoder attention unit may receive the output of the first masked attention unit and the encoder output generated by the last encoder 520-1 for the first time instant, and the first decoder feedforward unit may receive the output of the first decoder attention unit and generate a first decoder output for the second time instant.

For each of the second to last decoders 530-2 to 530-N, the masked attention unit of the decoder can receive the decoder output generated by the previous decoder, the decoder attention unit can receive the output of the masked attention unit and the encoder output generated by the last encoder 520-N for the first time instant, and the decoder feedforward unit can receive the output of the decoder attention unit and generate a corresponding decoder output for the second time instant.

The decoder output generated by the last decoder 530-N may be similar to the decoder output generated by the decoder 430 discussed above in conjunction with FIG. 4. As discussed above in conjunction with FIG. 4, the decoder output generated by the last decoder 530-N may be used as the model prediction value 504, or the model prediction value 504 may be generated by further processing the decoder output generated by the last decoder 530-N using the transformer module 510.

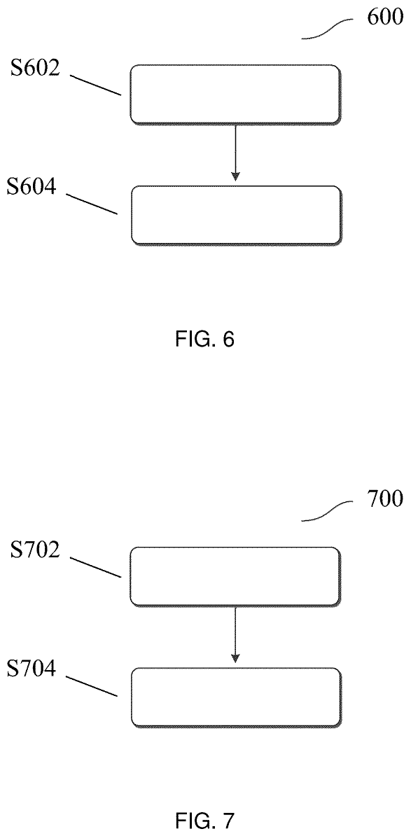

FIG. 6 shows an example flowchart of the method 600 of using a deep learning model to analyze multi-channel time series signals according to one example of the present disclosure.

At step S602, multi-channel time series signals may be obtained.

At step S604, a model prediction value can be generated based on the multi-channel time series signals using a deep learning model. The deep learning model may comprise a convolutional neural network module and a transformer module. The convolutional neural network module may be configured to receive the multi-channel time series signals and generate a convolutional output. The transformer module may be configured to receive a convolutional output and generate a model prediction value. In one example scenario, the multi-channel time series signals obtained in step S602 may comprise EEG signals. The EEG signals may be acquired from different positions of the head of a driver of a vehicle. In this example scenario, the model prediction value generated at step S604 may indicate the level of alertness of the driver. In this example scenario, the vehicle may be controlled according to the model prediction value. FIG. 7 shows an example flowchart 700 of a method for controlling a vehicle according to one example of the present disclosure.

At step S702, a model prediction value may be obtained. The model prediction value can be generated using the method discussed above in conjunction with FIG. 6 for analyzing the multi-channel time series signals using a deep learning model.

At step S704, instructions may be generated based on the model prediction value, which may be used to trigger the autonomous driving control unit of the vehicle to perform autonomous driving operations. For example, when it is identified based on the model prediction value that the driver is falling asleep or losing focus on the driving environment while driving, instructions can be generated to, for example, promptly activate various autonomous driving technologies to avoid dangerous situations.

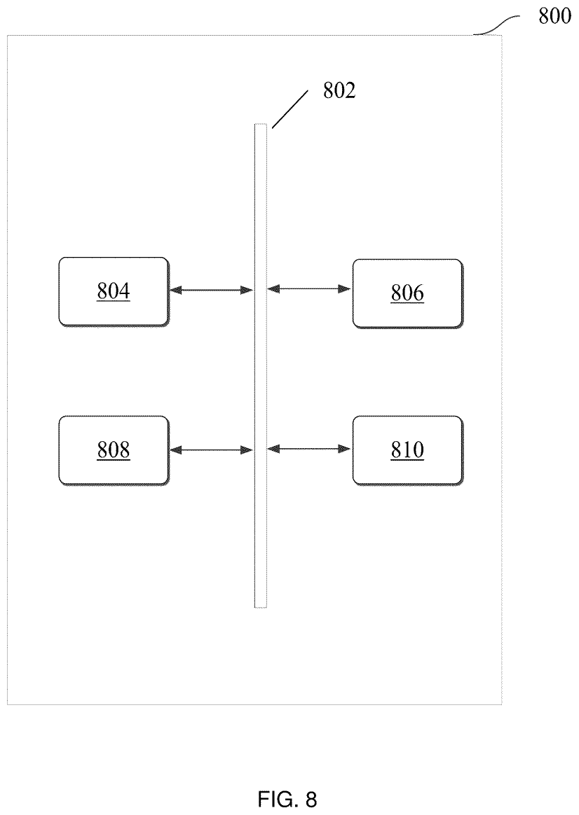

FIG. 8 shows a block diagram of a device 800 for processing multi-channel time series signals according to one example of the present disclosure. The device 800 may comprise any computing device on which the deep learning model discussed in the present disclosure may be deployed to generate a model prediction value based on multi-channel time series signals. In one example, such as for the example application scenario of performing the driver alertness estimation task discussed above, the device 800 may include a control unit for an autonomous driving vehicle, such as a vehicle control unit (VCU), an electronic control unit (ECU), and so on. In such an example, the device 800 may utilize a deep learning model to analyze the driver's EEG signals in real time and generate a model prediction value indicative of the driver's alertness level, and may further generate instructions to trigger the autonomous driving control unit of the vehicle to perform autonomous driving operation based on the model prediction value.

The example device 800 comprises a processor 804 connected to an internal communication bus 802 for executing instructions in the memory 806 to implement the method for analyzing multi-channel time series signals with the deep learning model detailed above. Examples of the processor 804 may comprise a central processing unit (CPU), a microcontroller, etc. The memory 806, which is suitable for tangibly embodying computer program instructions and data, may comprise various forms of storage such as EPROM, EEPROM, flash memory devices, and so on. The device 800 may further comprise an input interface 808 and an output interface 810. The input interface 808 may be used to receive input signals and data, such as the multi-channel time series signals discussed above. The output interface 810 may be used to send output signals and data, such as the model prediction value discussed above or instructions to trigger the autonomous driving control unit of the vehicle to perform autonomous driving operations based on the model prediction value.

The computer program may include instructions executable by a computer for causing the processor 804 of the device 800 to execute the method of the present disclosure for analyzing multi-channel time series signals using a deep learning model. The program may be recorded on any data storage medium, including the memory. For example, the program may be implemented in digital electronic circuits or using computer hardware, firmware, software, or a combination thereof. The process/method steps described in the present disclosure can be performed by a programmable processor executing program instructions to operate on input data and generate output to perform the method steps, processes, operations.

Embodiments of the present disclosure may be implemented in a computer-readable medium. The computer-readable medium may store a computer program comprising instructions. In one example aspect, the instructions, when executed by the processor, may cause the processor to: obtain the multi-channel time series signals; and use the deep learning model to generate a model prediction value based on the multi-channel time series signals; wherein: the deep learning model comprises a convolutional neural network module and a transformer module; the convolutional neural network module is configured to receive the multi-channel time series signals and generate a convolutional output; and the transformer module is configured to receive the convolutional output and generate the model prediction value. In another example aspect, the instructions, when executed by the processor, may cause the processor to: obtain a model prediction value generated according to the above analysis method; and generate instructions based on the model prediction value for triggering an autonomous driving control unit of the vehicle to perform an autonomous driving operation.

Embodiments of the present disclosure may be implemented in a computer program product. The computer program product may include instructions. In one example aspect, the instructions, when executed, may cause the one or more processors to: obtain the multi-channel time series signals; and use the deep learning model to generate a model prediction value based on the multi-channel time series signals; wherein: the deep learning model comprises a convolutional neural network module and a transformer module; the convolutional neural network module is configured to receive the multi-channel time series signals and generate a convolutional output; and the transformer module is configured to receive the convolutional output and generate the model prediction value. In another example aspect, the instructions, when executed, may cause the one or more processors to: obtain a model prediction value generated according to the above analysis methods; and generate instructions based on the model prediction value for triggering the autonomous driving control unit of the vehicle to perform autonomous driving operations

In addition to the content described in this document, various modifications can be made to the disclosed examples and implementations of the present disclosure without departing from the scope of the disclosed examples and examples of the present disclosure. Therefore, the description and examples herein should be interpreted as illustrative and not restrictive. The scope of the present disclosure should only be determined by reference to the claims.

What is claimed is:

1. A method for analyzing multi-channel time series signals using a deep learning model, comprising:
obtaining the multi-channel time series signals; and
using the deep learning model to generate a model prediction value based on the multi-channel time series signals,
wherein the deep learning model comprises a convolutional neural network module and a transformer module,
wherein the convolutional neural network module is configured to receive the multi-channel time series signals and generate a convolutional output, and
wherein the transformer module is configured to receive the convolutional output and generate the model prediction value.

2. The method according to claim 1, wherein the convolutional neural network module comprises at least one convolutional layer and at least one corresponding pooling layer arranged alternately.

3. The method according to claim 2, wherein the convolutional neural network module is further configured to:
shape the multi-channel time series signals to generate shaped two-dimensional input data; and
the shaped two-dimensional input data is used as an input to the first convolutional layer of the at least one convolutional layer.

4. The method according to claim 2, wherein the convolutional neural network module is further configured to:
shape an output of the last pooling layer of the at least one pooling layer to generate a one-dimensional convolutional output.

5. The method according to claim 1, wherein the convolutional output comprises a series of convolutional values corresponding to a plurality of time instants.

6. The method according to claim 5, wherein the transformer module comprises at least one encoder and at least one corresponding decoder.

7. The method according to claim 6, wherein the transformer module comprises an encoder and a decoder, the encoder comprising:
an encoder attention unit configured to receive a convolutional value corresponding to a first time instant of the plurality of time instants in the convolutional output; and
an encoder feedforward unit configured to receive an output of the encoder attention unit and generate an encoder output for the first time instant.

8. The method according to claim 7, wherein the decoder further comprises:
a masked attention unit configured to receive a convolutional value corresponding to a second time instant of the plurality of time instants in the convolutional output, wherein the first time instant is prior to the second time instant;
a decoder attention unit configured to receive an output of the masked attention unit and an encoder output for the first time instant; and
a decoder feedforward unit configured to receive an output of the decoder attention unit and generate a decoder output for the second time instant.

9. The method according to claim 6, wherein, if the transformer module comprises a plurality of encoders and a plurality of corresponding decoders:
the plurality of encoders are connected in series; and
the plurality of decoders are connected in series.

10. The method according to claim 9, wherein:
the first encoder of the plurality of encoders uses the convolutional output as input and generates a first encoder output, and each encoder from the second encoder to the last encoder of the plurality of encoders uses the encoder output generated by the previous encoder as input and generates a corresponding encoder output; and
the first decoder of the plurality of decoders uses the convolutional output and the encoder output generated by the last encoder as input and generates a first decoder output, and each decoder from the second decoder to the last decoder of the plurality of decoders uses the encoder output generated by the last encoder and the decoder output generated by the previous decoder as input and generates a corresponding decoder output.

11. The method according to claim 10, wherein:
each encoder of the plurality of encoders comprises an encoder attention unit and an encoder feedforward unit; and
each encoder of the plurality of decoders comprises a masked attention unit, a decoder attention unit, and a decoder feedforward unit.

12. The method according to claim 11, wherein:
the encoder attention unit of the first encoder is configured to receive a convolutional value corresponding to the first time instant of the plurality of time instants in the convolutional output;
the encoder attention unit of each encoder from the second encoder to the last encoder is configured to receive the encoder output generated by the previous encoder; and
the encoder feedforward unit of each encoder is configured to receive the output of the encoder attention unit of the encoder and generate a corresponding encoder output for the first time instant.

13. The method according to claim 12, wherein:
the masked attention unit of the first decoder is configured to receive a convolutional value corresponding to a second time instant of the plurality of time instants in the convolutional output, wherein the first time instant is prior to the second time instant;
the masked attention unit of each decoder from the second decoder to the last decoder is configured to receive the decoder output generated by the previous decoder;
the decoder attention unit of each decoder is configured to receive the output of the masked attention unit of the decoder and the encoder output generated by the last encoder for the first time instant; and
the decoder feedforward unit of each decoder is configured to receive the output of the decoder attention unit of the decoder and generate a corresponding decoder output for the second time instant.

14. The method according to claim 8, further comprising:
generating, by the transformer module, a model prediction value for the second time instant based on the decoder output generated by the decoder or by the last decoder for the second time instant.

15. The method according to claim 1, wherein:
the multi-channel time series signals comprise EEG signals; and
the EEG signals are acquired from different positions of the head of a driver of a vehicle.

16. The method according to claim 15, wherein the model prediction value is used to indicate a level of alertness of the driver.

17. A method for controlling a vehicle, comprising:
obtaining a model prediction value, wherein the model prediction value is generated using the method according to claim 1; and
generating instructions for triggering an autonomous driving control unit of the vehicle to perform an autonomous driving operation based on the model prediction value.

18. A device for processing multi-channel time series signals, comprising:
a memory; and
a processor coupled to the memory, the processor being configured to perform the method according to claim 1.

19. A computer-readable medium storing a computer program comprising instructions, the instructions, when executed by the processor, causing the processor to be configured to perform the method according to claim 1.

20. A computer program product comprising computer-executable instructions that, when executed, cause one or more processors to perform the method according to the method of claim 17.
